(19) United States
(12) Patent Application Publication
(10) Pub. No.: US 2022/0115103 A1
(43) Pub. Date: Apr. 14, 2022

(54) CONSUMPTION WEARABLE

(71) Applicant: Radient Technologies Innovations Inc., Edmonton (CA)

(72) Inventors: Michael Cabigon, Edmonton (CA); Jim Seethram, Edmonton (CA); Steven Splinter, Edmonton (CA); Denis Taschuk, Edmonton (CA)

(21) Appl. No.: 17/239,893

(22) Filed: Apr. 26, 2021

Related U.S. Application Data
(63) Continuation of application No. PCT/IB2019/058962, filed on Oct. 22, 2019.
(60) Provisional application No. 62/750,142, filed on Oct. 24, 2018.
(60) Provisional application No. 62/750,207, filed on Oct. 24, 2018.

(51) Int. Cl. G16H 20/10; G16H 40/67; G16H 10/60

(52) U.S. Cl. CPC G16H 20/10; G16H 10/60; G16H 40/67

(57) ABSTRACT

A system and method is provided that includes a wearable device that measures the effects of cannabis products on users based on an algorithm that calculates biometric data received by sensors on the device. The system includes a delivery device that has a wireless connection to a third-party network that monitors their usage and compares the usage data to the use prescribed in the user's treatment plan. When the user exceeds their treatment plan the network will remotely deactivate the user's delivery device. The system will also notify the user when they have not met the minimum usage prescribed by their treatment plan. The system further provides a means for caregivers to monitor the patient's usage and remotely lock or unlock the user delivery device. A method of monitoring cannabis usage in memory impaired patients to ensure compliance with a treatment plan is provided.

### CROSS-REFERENCED TO RELATED APPLICATIONS

The present patent application is a continuation of International Application No. PCT/IB2019/058962 filed Oct. 22, 2019, which claims the priority benefit of U.S. provisional patent No. 62/750,142 filed Oct. 24, 2018 and U.S. provisional patent No. 62/750,207 filed Oct. 24, 2018, the disclosures of which are incorporated by reference herein.

### BACKGROUND OF THE INVENTION

### 1. Field of the Invention

The present disclosure is generally related to monitoring cannabis consumption. More particularly, the present disclosure relates to monitoring cannabis consumption in patients with memory issues.

### 2. Description of the Related Art

Cannabis is a genus belonging to the family of cannabaceae. Three common species include Cannabis sativa, Cannabis indica, and Cannabis ruderalis. Cannabis has a long history being used for medicinal, therapeutic, and recreational purposes. Historical delivery methods for cannabis have involved smoking (e.g., combusting) the dried cannabis plant material. Alternative delivery methods such as ingesting typically require extracts of the cannabis biomass (i.e., cannabis concentrates or cannabis oils). Often, cannabis extracts are formulated using any convenient pharmacologically or food-grade acceptable diluents, carriers or excipients to produce a composition, which collectively may be known as cannabis derivative products or cannabis products. These may for example include cannabis topicals, edibles, or vaping products. Cannabis edibles may include cannabis-infused food or beverage products.

The current state of the art in vaporized medication compliance systems only look for the upper limit of what a user is allowed or should be using and delivers a notification when that limit is reached. This is because they are focused on nicotine replacement therapy. With cannabis products, and in particular for patients with memory issues, it is important to ensure patients both use the minimum amount required and do not exceed the maximum.

Wearable devices are smart electronic devices (i.e., electronic devices with micro-controllers) that can be incorporated into clothing or worn on the body as implants or accessories. Wearable technology has a variety of applications which grows as the field itself expands. Wearable device's appear prominently in consumer electronics with the popularization of the smartwatch and activity tracker, but are slowing finding their way into healthcare.

### SUMMARY OF THE CLAIMED INVENTION

The present invention monitors user effects of a cannabis product on a user via an apparatus that monitors the effects of cannabis products by measuring the user's biometric performance, such as heart rate, skin temperature, blood pressure, etc. In some embodiments, the apparatus prompts the user to perform a quick test on the user application to determine the post-consumption effects of the cannabis product. A method is provided to treat users with memory disorders by reminding the user when to consume cannabis products and keep the user's cannabis product consumption within acceptable limits.

### BRIEF DESCRIPTIONS OF THE DRAWINGS

FIG. 1 illustrates an exemplary network environment in which a wearable device for monitoring cannabis consumption or treatment may be monitored.

FIG. 2 is a flowchart illustrating an exemplary method of dosage optimization.

FIG. 3 is a flowchart illustrating an exemplary method of dosage monitoring.

FIG. 4 is a flowchart illustrating an exemplary method of dosage recommendation.

FIG. 5 is a flowchart illustrating an exemplary method of monitoring usage in accordance with a treatment plan.

FIG. 6 is a flowchart illustrating an exemplary method of treatment adjustment.

### DETAILED DESCRIPTION

Embodiments of the present invention include systems and methods for monitoring cannabis usage and treatment. Such systems may include a wearable device that measures the effects of cannabis products on users based on an algorithm that calculates biometric data received by sensors on the device. The system includes a delivery device (e.g. vaporizing device) that has a wireless connection to a third-party network that monitors their usage and compares the usage data to the use prescribed in the user's treatment plan. When the user exceeds their treatment plan the network will remotely deactivate the user's delivery device. The system will also notify the user when they have not met the minimum usage prescribed by their treatment plan. The system further provides a means for caregivers to monitor the patient's usage and remotely lock or unlock the user delivery device. A method of monitoring cannabis usage in memory-impaired patients to ensure compliance with a treatment plan is provided.

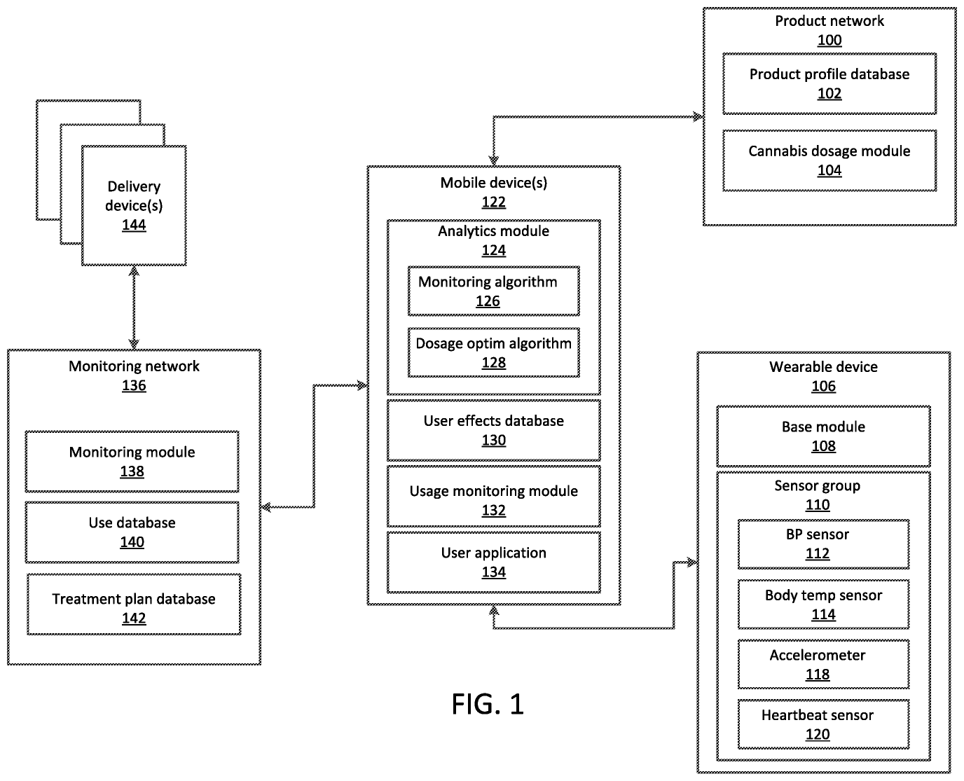

FIG. 1 illustrates an exemplary network environment in which a wearable device for monitoring cannabis consumption or treatment may be monitored. For the purposes of this invention, cannabis or “cannabis product” may mean any product containing cannabis in any delivery form. The system comprises a product network 100 that contains all the cannabis concentrate or product profile data, looks up the product being used by the consumer, and sends the concentrate or product profile (e.g. concentration of cannabinoids such as THC or CBD, amount of cannabinoid delivered per discrete unit of product (i.e., the dosage), cannabinoid profile, terpene profile, etc.) as input data to the analytics module on the mobile device to run the dosage optimization algorithm. A product profile database 102 contains all the cannabis products and the composition and dosage in each.

A cannabis dosage module 104 is a software module that retrieves from the product profile database 102 the profile data of the cannabis concentrate or product being consumed and sends the profile data to an analytics module 124 on a mobile device 122 (e.g., laptop, desktop, tablet, smartphone, smartwatch, etc.). The cannabis dosage module 104 looks up the cannabis product being consumed in the product profile database 102 and retrieves the cannabis product profile data. The cannabis dosage module 104 then calculates the dosage of cannabis in the product and sends the concentrate profile data to the analytics module 124 on the mobile device 122 for further calculations.

A wearable device 106 gathers the biometric data of the user using a group of sensors 110 (blood pressure sensor 112, body temperature sensor 114, accelerometer 116, heartbeat sensor 118), and sends the biometric data to the mobile device 122 through a communication path. A base module 108 is a software module that activates the sensor group 110 and communication path, or in some embodiments, a selected group of sensors. It should allow different “modes” that require various combinations of sensors. The base module 108 activates the sensor group 110 upon the user's command and sends the biometric data through the communication path to the mobile device 122. In some embodiments, the activation of sensors can is based on mode selection.

The sensor group 110 is a group of sensors that collects biometric data of the user when activated. The sensor group 110 determines which “mode” is being selected and turns on the selected sensors. The sensor group 110 monitors the biometrics of the user during consumption and periodically sends the biometric data to the mobile device 122 through the communication path. The BP sensor 112 is a sensor that detects the blood pressure of the user. A body temperature sensor 114 a sensor that detects the body temperature of the user. An accelerometer 118 is a sensor that detects the movement of the user. A heartbeat sensor 120 is a sensor that detects the heartbeat rate of the user. A communication path (e.g. Bluetooth, Wi-Fi, NFC, or some other method) can communicate the biometrics data gathered by the sensor group 110 with the analytics module 124 on the mobile device 122.

The mobile device 122 allows the user to get the profile data of the cannabis product they consume, to be monitored during the consumption and receive recommendations, alerts or warnings based on the user's biometric data gathered by the wearable device 106 and the dosage data entered by the user. In some embodiments, the user can enter doctors' recommendation into the analytics module 124 through a user application 134 to get more accurate recommendations. The user application 134 can be a smart phone application that enables the user to enter the amount and type of product being consumed and displays the dosage recommendations and alerts to the user. In some embodiments, the user can look up the effects each type of product has on him/her based on biometric data, track long-term changes in user effects and compare the user effects of similar products. The user application 134 prompts the user to enter the amount and type of product being consumed and sends the cannabis concentrate or product type to the cannabis dosage module 104 in the product network 100. The user application 134 receives recommendations, alerts or warnings from the analytics module 124 and displays a message to the user.

The analytics module 124 is a software module that monitors the user effects during the consumption of cannabis and makes optimized recommendation on dosage of cannabis based on biometrics data and user input. The monitoring algorithm 126 is an algorithm that monitors the effects of cannabis consumption on the user based on the biometric data collected by the sensor group 110 on the wearable device 106, notifies the changes during and after consumption and sends the processed data to the dosage optimization algorithm 128, and also stores the user effects data on the user effects database 130. The dosage optimization algorithm 128 is an algorithm that optimizes the cannabis dosage based on the user input data and the user effects reflected by the processed biometric data. The user effects database 130 is a database that stores the user ID, the cannabis concentrate or product consumed by the user and the biometric data associated with the user's consumption. Biometric data includes the data from the blood pressure (BP) sensor, body temperature sensor (Celsius), accelerometer (m/s\hat{ }2) and heartbeat sensor (bpm).

| | Cannabis | | Body | | |
|---|---|---|---|---|---|
| User | Concentrate/ | BP | Temp | Acceler- | Heartbeat |
| Consumption | Product | Sensor | Sensor | ometer | Sensor |
| 1 | Ringo's | 130/90 | 37 | 1 | 75 |
| 2 | Sweet and Sour | 120/80 | 38 | 0 | 60 |
| 3 | Cannatonic | 140/90 | 36.7 | 35 | 78 |

One or more mobile device(s) 122 can communicate with a monitoring network 136 and deliver notifications to the user. The mobile device 122 can communicate with the monitoring network 136 to receive notifications of user usage patterns and make decisions about authorizing use and/or remotely locking or unlocking a user delivery device 144. A usage monitoring module 132 allows the caregiver to remotely view their patient's use levels and notify them if they are below their treatment plans called for intake, as well as lock their device if they have exceeded their usage limits or unlock their locked device if they caregiver feels they need to exceed their treatment plan.

One or more user delivery device(s) 144 can be utilized by the user to administer the cannabis product. The delivery device 144 can be a number of different devices but will need to include at least; a communications unit for communicating usage data to an external database as well as a means of remotely deactivating the activator switch on the unit for a specific time period. The monitoring network 136 provides centralized storage of patient/user usage data to caregivers, as well as providing a means to remotely interact with the user delivery device 144 based on the user's usage. A monitoring module 138 compares usage of the user delivery device 144 with a treatment plan database 142 data to automatically notify the user when they have forgotten to take their recommended dosage and deactivate the user delivery device 144 without caregiver intervention when the user exceeds their treatment plan. A use database 140 stores all the use data supplied by the user delivery device(s) 144 connected to the system. The treatment plan database 142 contains the recommended use level and period for each user in the system.

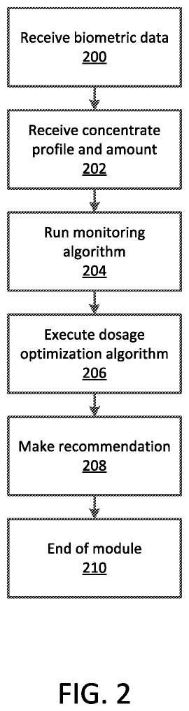

FIG. 2 is a flowchart illustrating an exemplary method of dosage optimization. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The process begins with at step 200 the analytics module 124 receiving biometric data from the mobile device 122. At step 202, the analytics module 124 receives the concentrate profile data from the cannabis dosage module 104 and the amount of cannabis concentrate or product being consumed from user input on the user application 134. At step 204, the analytics module 124 runs the monitoring algorithm 126. At step 206, the analytics module 124 executes the dosage optimization algorithm 128 based on the concentrate profile data, the amount of cannabis concentrates or product being consumed, and the biometric data. At step 208, the analytics module 124 formulates a recommendation, alert or warning to the user application 134.

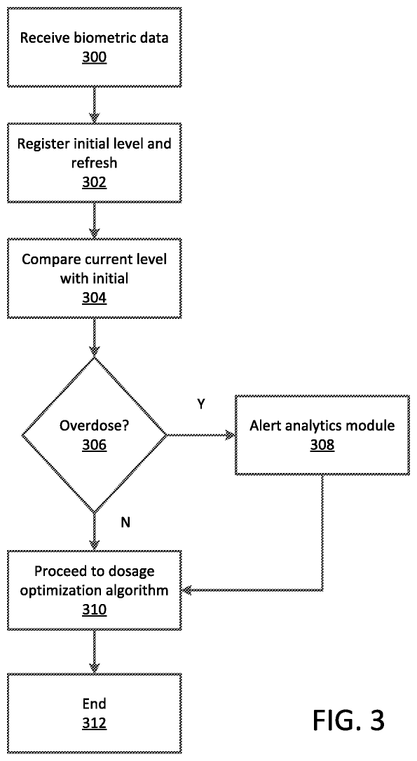

FIG. 3 is a flowchart illustrating an exemplary method of dosage monitoring. The process begins at step 300 when the monitoring algorithm 126 receives the biometric data from the mobile device 122. At step 302, the monitoring algorithm registers the initial biometric performance level and refreshes the biometric data every set period in the user effects database 130. At step 304, the monitoring algorithm 126 compares the current biometric performance level with the initial level every set period and registers the changes in the user effects database 130. For example, the wearable device 106 would know the movement, temperature, pulse of the user as well as when the cannabis device is on. A baseline is established when no cannabis is used. The baseline is averaged during time periods during the day. When cannabis is used, sensors provide data related to movement, temperature and pulse are collected. The first comparison is any of the sensor data/parameters that may be out of range. This would provide a warning or an alert, with the annotation that it is likely cannabis use. If the sensor data/parameters say activity is less that the average during the same time period, this method step may show the user is lethargic (i.e., slow movements) possibly due to cannabis consumption. A recommendation may be to stop using the cannabis device until normal movement is detected. The level of potency can also be correlated to the same time usage, but with movement at the same time period. For each of the sensor data, the average nonuse data is compared to the sensor data in use for time and potency of the cannabis, where recommendations can be made to consume less cannabis. Also, the combined sensor data can be cross-correlated with each other to find patterns. For example, pulse may drop first, then temperature drops, then movement slows down, this pattern could be different than the normal average for the same period, so this pattern could be a correlated pattern to the onset of cannabis use and pointed out to the user that the onset effects have started. Likewise, the disappearance of affects can be correlated to tell the user the disappearance of affects has started. At step 606, the monitoring algorithm 126 determines if there has been an overdose. If there was an overdose, then the monitoring algorithm 126 formulates an alert or warning to the analytics module 124 at step 308. At step 310, the monitoring algorithm 126 proceeds to the dosage optimization algorithm 128. At step 312, the monitoring algorithm 126 ends.

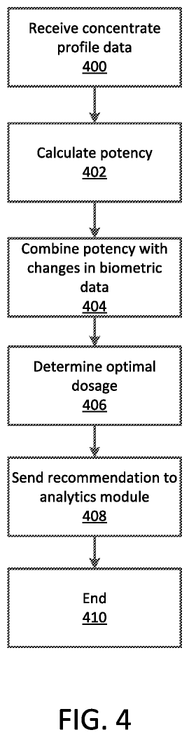

FIG. 4 is a flowchart illustrating an exemplary method of dosage recommendation. The process begins at step 400 with the dosage optimization algorithm 128 receiving concentrate profile data from the analytics module 124 on the potency content and concentration in the product. At step 402, the dosage optimization algorithm 128 calculates the quantity of potency content based on the data received. At step 404, the dosage optimization algorithm 128 combines the quantity of potency content with the monitored changes in biometric data. At step 406, the dosage optimization algorithm 128 determines the optimal dosage of cannabis concentrate or product for the user. At step 408, the dosage optimization algorithm 128 sends the recommendation, alert or warning to the analytics module 124.

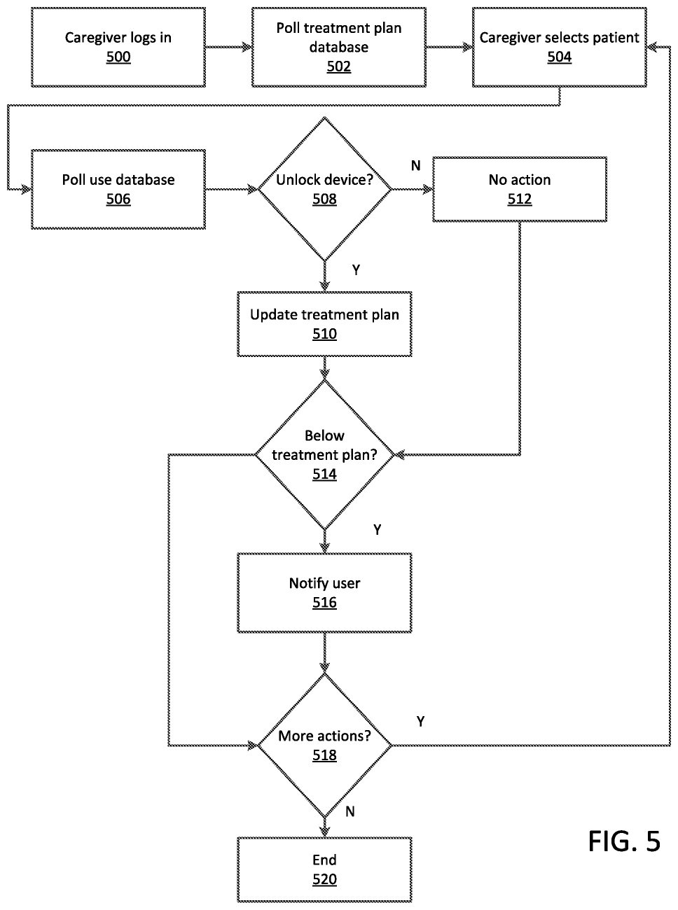

FIG. 5 is a flowchart illustrating an exemplary method of monitoring usage in accordance with a treatment plan. The process begins at step 500 when the caregiver logs into the usage monitoring module 132 on the caregiver mobile device 122. At step 502, the usage monitoring module 132 polls the treatment plan database 142 for patient(s) logged in the system that the caregiver is an authorized caregiver for and presents them in a selectable format to the caregiver. At step 504, the caregiver selects the patient whom they wish to view usage data for. At step 506, the usage monitoring module 132 polls the use database 140 for the selected patient's use history. At step 508, the usage monitoring module 132 determines if the user delivery device 144 is currently locked and whether the caregiver wants to unlock it. If the user delivery device 144 is locked and the caregiver wants to unlock it, the usage monitoring module 132 updates the treatment plan for the user at step 510. For example, if a user's treatment plan calls for between 30 and 50 mg of THC every eight hours, and the user has exceeded 50 mg in the current eight-hour window, the caregiver can increase the treatment plan range to 40-60 mg. This change is then reflected in the treatment plan database 142 so that when the monitoring module 138 checks the treatment plan database 142, the monitoring module 138 will unlock the user delivery device 144 on the user's next attempt to intake THC, because the user will be under their new treatment plan limits. If the caregiver does not wish to unlock the device at that time, no action is taken at step 512. At step 514, the usage monitoring module 132 determines if the user is below the amount of THC, or other cannabinoid, the user's treatment plan calls for them to consume. At step 516, the usage monitoring module 132 sends a notification to the user mobile device 122 that the user is below their treatment plans called for intake. In another embodiment, the usage monitoring module 132 can send a notification to the user delivery device 144. For example, the user's treatment plan calls for between 30 and 50 mg of THC every eight hours, and the user has only consumed 10 mg in the current eight-hour window. In that situation the user would get a reminder on their mobile device 122 that their treatment plan calls for the intake of at least 20 more mg before the end of the eight-hour window. The usage monitoring module 132 asks the caregiver if they would like to take more actions at step 518. For example, if the caregiver has multiple patients under the caregiver's care in this same manner, they can go back to the list presented in 504 to select another patient. In another embodiment, the caregiver can lock a patient's device, even when they have not reached treatment plan limits, by adjusting the treatment plan down to an amount below the user's current consumption level. Otherwise, the module ends at 520.

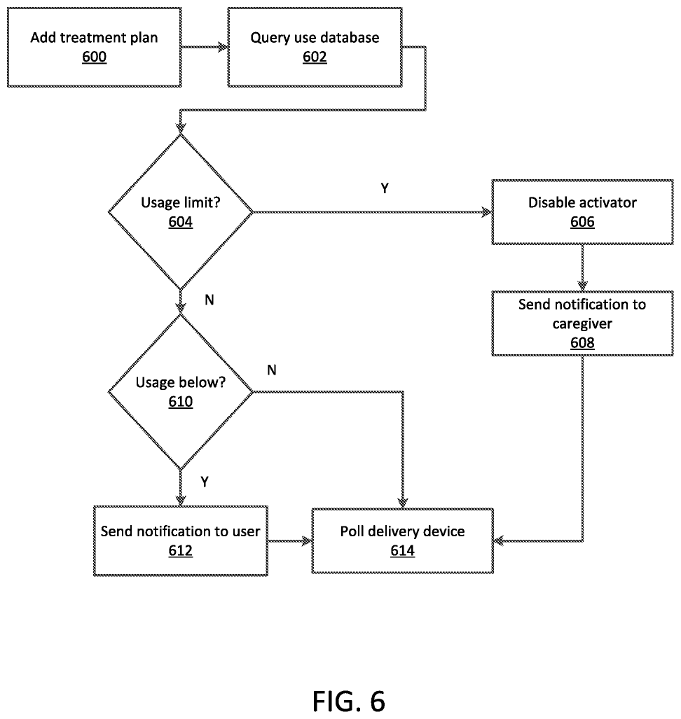

FIG. 6 is a flowchart illustrating an exemplary method of treatment adjustment. The process begins at step 600 with a user's treatment plan being added to or altered in the treatment plan database 142. At step 602, the monitoring module 138 queries the use database 140 for use data associated with the user. At step 604, the monitoring module 138 determines if the user has reached their usage limit. If the user has reached their usage limit and exceeded the use prescribed in their treatment plan, the monitoring module 138 disables the activator on the user delivery device 144 at step 606. At step 608, the monitoring module 138 sends a notification to the caregiver's mobile device 122 that the user has exceeded their usage limit for a given time. This allows the caregiver to open a communications channel with the user to determine if the user's treatment plan needs to be adjusted. At step 610, the monitoring module 138 determines if the user is below the recommended usage level for the current time according to the treatment plan. If the user is not below the recommended usage level, the process moves to step 614. If the user is below the recommended usage level, the monitoring module 138 sends a notification to the user device to remind the user of the amount of product they still must administer before the end of the current time period according to their treatment plan at step 612. At step 614, the monitoring module 138 polls the user delivery device 144 for a new usage data point and returns to step 600 when a new usage data point is detected.

The foregoing detailed description of the technology has been presented for purposes of illustration and description. It is not intended to be exhaustive or to limit the technology to the precise form disclosed. Many modifications and variations are possible in light of the above teaching. The described embodiments were chosen in order to best explain the principles of the technology, its practical application, and to enable others skilled in the art to utilize the technology in various embodiments and with various modifications as are suited to the particular use contemplated. It is intended that the scope of the technology be defined by the claims.

What is claimed is:

1. A method for monitoring user effects of cannabis consumption, the method comprising:
receiving profile data for a product from a product profile database;
receiving biometric data from one or more sensors on a wearable device worn by a user;
receiving input regarding consumption of a cannabis product by the user;
formulating a recommendation regarding a dosage of the cannabis product for the user based on the profile of the cannabis product and the biometric data; and
presenting the recommendation via a user device.

2. The method of claim 1, wherein the sensors include at least one of a blood pressure sensor, a body temperature sensor, an accelerometer, and a heartbeat sensor.

3. The method of claim 1, further comprising receiving a sensor selection from the user, wherein the biometric data is received from the sensors in the sensor selection.

4. The method of claim 3, further comprising activating the sensors in the sensor selection.

5. The method of claim 1, further comprising:
polling a use database on a monitoring network for usage data about the user; and
receiving the usage data about the user from the use database, wherein the recommendation is further based on the usage data.

6. The method of claim 5, wherein the usage data in the use database is supplied by one or more user delivery devices that administer one or more cannabis products to the user.

7. The method of claim 6, further comprising:
evaluating the usage data about the user against a treatment plan, wherein the treatment plan specifies a recommended use level and is stored in a treatment plan database on the monitoring network; and
sending a notification to the user device if the usage data deviates from the treatment plan by a threshold amount.

8. The method of claim 7, wherein the usage data indicates a dosage that exceeds the treatment plan, and further comprising deactivating a user delivery device of the user.

9. The method of claim 7, further comprising sending a notification to a designated caregiver device that is able to remotely lock and unlock the user delivery device of the user.

10. A system for monitoring user effects of cannabis consumption, the system comprising:
a communication network interface that communicates over a communication network, wherein the communication interface:
receives profile data for a product from a product profile database, and
receives biometric data from one or more sensors on a wearable device worn by a user;
a user interface that receives input regarding consumption of a cannabis product by the user;
a processor that executes instructions stored in memory, wherein execution of the instructions by the processor formulates a recommendation regarding a dosage of the cannabis product for the user based on the profile of the cannabis product and the biometric data; and
an output device that presents the recommendation.

11. The system of claim 10, wherein the sensors include at least one of a blood pressure sensor, a body temperature sensor, an accelerometer, and a heartbeat sensor.

12. The system of claim 10, wherein the user interface further receives a sensor selection from the user, wherein the biometric data is received from the sensors in the sensor selection.

13. The system of claim 12, further comprising a base module executable to activate the sensors in the sensor selection.

14. The system of claim 10, further comprising a monitoring network device that:
polls a use database on a monitoring network for usage data about the user; and
receives the usage data about the user from the use database, wherein the recommendation is further based on the usage data.

15. The system of claim 14, wherein the usage data in the use database is supplied by one or more user delivery devices that administer one or more cannabis products to the user.

16. The system of claim 15, wherein the processor further evaluates the usage data about the user against a treatment plan, wherein the treatment plan specifies a recommended use level and is stored in a treatment plan database on the monitoring network; and wherein the communication network interface further sends a notification to the user device if the usage data deviates from the treatment plan by a threshold amount.

17. The system of claim 16, wherein the usage data indicates a dosage that exceeds the treatment plan, and wherein the processor further deactivates a user delivery device of the user.

18. The system of claim 17, wherein the communication network interface further sends a notification to a designated caregiver device that is able to remotely lock and unlock the user delivery device of the user.
